(19) United States
(12) Patent Application Publication
(10) Pub. No.: US 2009/0064998 A1
(43) Pub. Date: Mar. 12, 2009

(54) Breathing Tube Bite Inhibitor

(76) Inventor: Hosni R. Bassili, Houston, TX (US)

Correspondence Address: Dr. Hosni R. Bassili, 5110 San Felipe, Unit 351, Houston, TX, 77056, US

(21) Appl. No.: 11/852,647

(22) Filed: Sep. 10, 2007

(51) Int. Cl. A61M 16/01

(52) U.S. Cl. 128/200.26

(57) ABSTRACT

This invention relates to bite inhibitors comprising a flange and a bite restricting member for use with a patient intubated with a breathing tube and to the combination of a bite inhibitor and a breathing tube mounted to the bite inhibitor.

### FIELD OF THE INVENTION

This invention relates to bite inhibitors comprising a flange and a bite restricting member for use with a patient intubated with a breathing tube and to the combination of a bite inhibitor and a breathing tube mounted to the bite inhibitor.

### DESCRIPTION OF THE DRAWINGS

FIG. 1 is a front view of a first embodiment of the invention.

FIG. 2 is a side view of a first embodiment of the invention.

FIG. 3 is a top view of a first embodiment of the invention.

FIG. 4 is a front view of a second embodiment of the invention being used by a patient.

### DESCRIPTION OF THE PREFERRED EMBODIMENTS

During general anesthesia, oxygen and other gases are often delivered to a patient through a breathing tube, such as an endotracheal tube or a laryngeal mask airway. Patients often occlude the breathing tube by biting down on it. Such occlusion can stop gas delivery to the patient. This invention provides a bite inhibitor to prevent such occlusion of a breathing tube in an orally intubated patient.

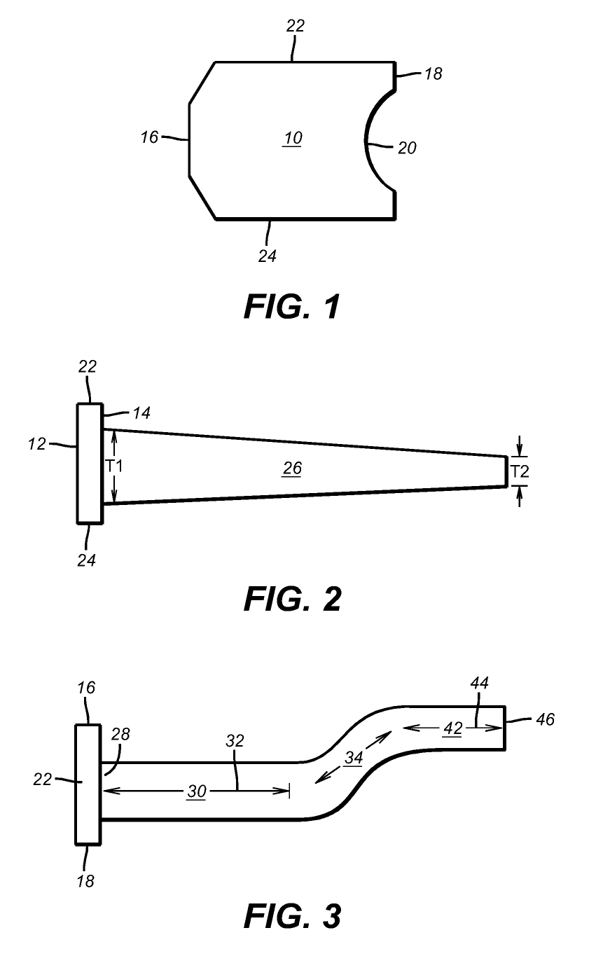

One embodiment of the invention is directed toward a bite inhibitor for use with a patient orally intubated with a breathing tube. This embodiment comprises a flange member 10 comprising a front surface 12, a rear surface opposite the front surface 14, an outer edge 16, an inner edge 18 opposite the outer edge and comprising a curved indentation 20 sized and shaped to snugly fit a breathing tube, as shown in FIGS. 1-3. The flange member further comprises a top edge 22, and a bottom edge 24 opposite the top edge and spaced from the top edge to define a height that exceeds the thickness of the lips of the patient, as shown in FIGS. 1, 2 and 4. In a preferred embodiment, the height of the flange is at least 1.0 inch. In a preferred embodiment, the flange is made from a nontoxic biocompatible material.

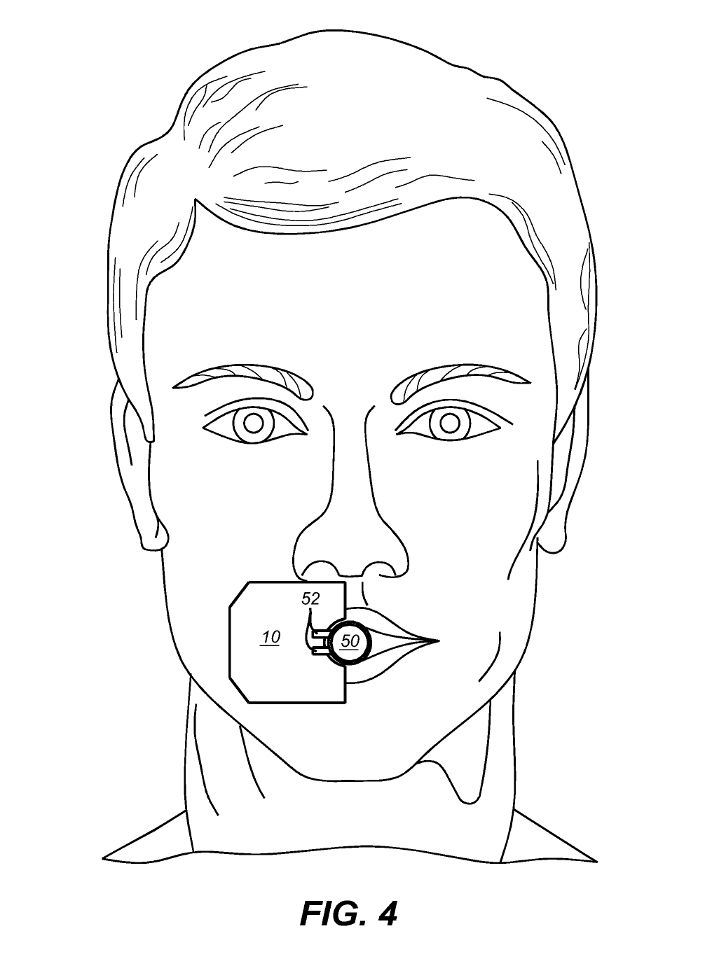

This embodiment of the invention further comprises a bite restricting member 26 made from a nontoxic biocompatible material, as shown in FIG. 2. In a preferred embodiment, the biocompatible material is a polymer. This bite restricting member comprises a front end 28 attached to the rear surface of the flange and a proximal section 30 extending rearward from the rear surface of the flange member to define a first longitudinal axis 32. The proximal section has a first thickness, T1, at its front end, as shown in FIGS. 2-3.

This bite restricting member further comprises a curved section 34 comprising a first end attached to the proximal section, a first curve section 36 curving outward, and a second curve section 38 curving rearward, as shown in FIG. 3. In a preferred embodiment, the curved section is sized to conform to the contour of the lateral side of a patient's jaw.

This bite restricting member further comprises a distal section 42 attached to the second curve section to define a second longitudinal axis 44 that is substantially parallel to the first longitudinal axis and offset outward of the first longitudinal axis, and a distal end 46 having a second thickness, T2, that is less than the first thickness, as shown in FIGS. 2-3. In a preferred embodiment, the thickness of the distal section is tapered. In a preferred embodiment, the second thickness is less than half of the first thickness. In another preferred embodiment, the outward offset is at least 0.25 inches.

Another embodiment of the invention is directed to a combination of the flange member and bite restricting member, as described above, and a breathing tube 50 mounted to the curved indentation and having a third thickness that is less than the first thickness, as shown in FIG. 4. In a preferred embodiment, the breathing tube is taped to the flange. Strips of tape 52 may be used to secure the tube to the flange, as shown in FIG. 4. In another preferred embodiment, the breathing tube is a laryngeal mask airway tube. In another preferred embodiment, the breathing tube is an endotracheal tube. When the invention is positioned in the patient's mouth, the patient's cheek can provide lateral support for the invention.

The foregoing disclosure and description of the inventions are illustrative and explanatory. Various changes in the size, shape, and materials, as well as in the details of the illustrative construction and/or a illustrative method may be made without departing from the spirit of the invention.

What is claimed is:

1. A bite inhibitor for use with a patient orally intubated with a breathing tube, comprising:
a. a flange member comprising a front surface, a rear surface opposite the front surface, an outer edge, an inner edge opposite the outer edge and comprising a curved indentation sized and shaped to snugly fit a breathing tube, a top edge, and a bottom edge opposite the top edge and spaced from the top edge to define a height that exceeds the thickness of the lips of the patient; and
b. a bite restricting member made from a nontoxic biocompatible material comprising:
i. a front end attached to the rear surface of the flange;
ii. a proximal section extending rearward from the rear surface of the flange member to define a first longitudinal axis, said proximal section having a first thickness at its first end;
iii. a curved section comprising a first end attached to the proximal section, a first curve section curving outward, and a second curve section curving rearward; and
iv. a distal section attached to the second curve section to define a second longitudinal axis that is substantially parallel to the first longitudinal axis and offset outward of the first longitudinal axis, and a distal end having a second thickness that is less than the first thickness.

2. The bite inhibitor of claim 1, wherein the biocompatible material is a polymer.

3. The bite inhibitor of claim 1, wherein the curved section is sized to conform to the contour of the lateral side of a patient's jaw.

4. The bite inhibitor of claim 1, wherein the second thickness is less than half of the first thickness.

5. The bite inhibitor of claim 1, wherein the thickness of the distal section is tapered.

6. The bite inhibitor of claim 1, wherein the outward offset is at least 0.25 inches.

7. The bite inhibitor of claim 1, wherein the height of the flange is at least 1.0 inch.

8. The bite inhibitor of claim 1, wherein the flange is made from a nontoxic biocompatible material.

9. The bite inhibitor of claim 1, further comprising a breathing tube mounted to the curved indentation and having a third thickness that is less than the first thickness.

10. The bite inhibitor of claim 9, wherein the breathing tube is taped to the flange.

11. The bite inhibitor of claim 9, wherein the breathing tube is a laryngeal mask airway tube.

12. The bite inhibitor of claim 9, wherein the breathing tube is an endotracheal tube.

13. A bite inhibitor for use with a patient orally intubated with a breathing tube, comprising:
a. a flange member comprising a front surface, a rear surface opposite the front surface, an outer edge, an inner edge opposite the outer edge and comprising a curved indentation sized and shaped to snugly fit a breathing tube, a top edge, and a bottom edge opposite the top edge and spaced from the top edge to define a height that exceeds the thickness of the lips of the patient; and
b. a bite restricting member made from a nontoxic biocompatible material comprising:
i. a front end attached to the rear surface of the flange;
ii. a proximal section extending rearward from the rear surface of the flange member to define a first longitudinal axis, said proximal section having a first thickness at its first end;
iii. a curved section comprising a first end attached to the proximal section, a first curve section curving outward, and a second curve section curving rearward; and
iv. a distal section attached to the second curve section to define a second longitudinal axis that is substantially parallel to the first longitudinal axis and offset outward of the first longitudinal axis, and a distal end having a second thickness that is less than the first thickness and is tapered.

14. The bite inhibitor of claim 13, wherein the curved section is sized to conform to the contour of the lateral side of a patient's jaw.

15. The bite inhibitor of claim 13, wherein the second thickness is less than half of the first thickness.

16. The bite inhibitor of claim 13, wherein the outward offset is at least 0.25 inches.

17. The bite inhibitor of claim 13, further comprising a breathing tube mounted to the curved indentation and having a third thickness that is less than the first thickness.

18. A bite inhibitor for use with a patient orally intubated with a breathing tube, comprising:
a. a flange member made from a nontoxic biocompatible material, and comprising a front surface, a rear surface opposite the front surface, an outer edge, an inner edge opposite the outer edge and comprising a curved indentation sized and shaped to snugly fit a breathing tube, a top edge, and a bottom edge opposite the top edge and spaced from the top edge to define a height that exceeds the thickness of the lips of the patient; and
b. a bite restricting member made from a nontoxic biocompatible material comprising:
i. a front end attached to the rear surface of the flange;
ii. a proximal section extending rearward from the rear surface of the flange member to define a first longitudinal axis, said proximal section having a first thickness at its first end;
iii. a curved section comprising a first end attached to the proximal section, a first curve section curving outward, and a second curve section curving rearward; and
iv. a distal section attached to the second curve section to define a second longitudinal axis that is substantially parallel to the first longitudinal axis and offset outward of the first longitudinal axis, and a distal end having a second thickness that is less than the first thickness.

19. The bite inhibitor of claim 18, wherein the biocompatible material is a polymer.

20. The bite inhibitor of claim 18, wherein the height of the flange is at least 1.0 inch.
